(19) United States
(12) Patent Application Publication
(10) Pub. No.: US 2013/0030049 A1
(43) Pub. Date: Jan. 31, 2013

(54) METHODS OF TREATING DERMATOLOGICAL DISORDERS OR CONDITIONS

(71) Applicant: Dermira (Canada), Inc., Redwood City, CA (US)

(72) Inventors: David W.C. HUNT, Surrey (CA); Jerome Arthur MORRIS, Fort Collins, CO (US); Huijun JIANG, Vancouver (CA); Danny LEUNG, Coquitlam (CA); Ronald Erwin BOCH, North Vancouver (CA)

(73) Assignee: Dermira (Canada), Inc., Redwood City, CA (US)

(21) Appl. No.: 13/649,926

(22) Filed: Oct. 11, 2012

Related U.S. Application Data
(62) Division of application No. 12/513,094, filed on Mar. 9, 2010, now abandoned.
(60) Provisional application No. 60/856,604, filed on Nov. 3, 2006.

(51) Int. Cl. A61K 31/341; A61P 17/10; A61P 29/00

(52) U.S. Cl. 514/473

(57) ABSTRACT

This disclosure is directed to the use of TOFA in the treatment of inflammatory disorders, including dermatological disorders.

### FIELD OF THE INVENTION

This invention is directed to the use of 5-(tetradecyloxy)-2-furancarboxylic acid (TOFA) for the treatment of dermatological disorders or conditions characterized by sebaceous gland hyperactivity, such as acne and oily skin. This invention is also directed to pharmaceutical and dermatological compositions comprising TOFA for use in treating dermatological disorders or conditions characterized by sebaceous gland hyperactivity, such as acne and oily skin.

### BACKGROUND OF THE INVENTION

Hyperactive sebaceous gland disorders, such as acne vulgaris (acne), are common dermatological conditions affecting many people. Acne typically presents at the onset of puberty and peaks in incidence between 14 and 19 years of age. The prevalence of acne is greatly reduced by the middle of the third decade of life. Acne pathogenesis is multi-factorial involving sebaceous gland hyperactivity (increased production of sebum) with seborrhea, abnormal keratinocyte proliferation/desquamation and bacterial colonization promoting local inflammatory changes. As a consequence of the surge in androgen production at puberty, increased sebum production occurs along with abnormal desquamation of the epithelial lining of hair follicles. This mixture of sebum and cell debris is the basic ingredient of the comedone providing an ideal environment for the growth of Propionibacterium acnes (P. acnes), an anaerobic gram-positive bacterium that is part of normal skin flora and a key contributor to inflammatory acne. Bacterial-derived chemotactic factors and proinflammatory mediators subsequently foster local inflammatory reactions.

The clinical presentation of acne ranges from open comedones (whiteheads) and closed comedones (blackheads) for mild acne to the papules, pustules, nodules and cystic or mixed lesions for severe, inflammatory acne. Acne lesions typically occur on the face, upper back, chest and upper arms. The clinical course of acne tends to wax and wane. The severity of the condition is affected by multiple factors including seasonal and psychological influences as well as self-induced trauma by patients who habitually manipulate their lesions. Although generally transitory in course, moderate to severe inflammatory acne presents a true disease state that may cause long-term consequences for the subject including, but not limited to, socially disabling psychological damage and disfiguring physical scars.

A wide array of therapies for treating from moderate to severe acne is available. These therapies may affect specific aspects of the condition or in some cases affect several pathogenic factors. However, there are significant deficiencies in the currently available therapies for acne. Dermatological therapies are not fully effective against mild to moderate acne and many of the agents employed in these therapies produce skin irritation. Therapies employing dermatological retinoids and benzoyl peroxide are effective against mild to moderate acne by removing comedones, killing bacteria and/or reducing inflammation. Therapies employing antibiotics, given either dermatologically or orally, may be used to treat mild to moderate acne through the antibiotics' bacteriostatic and anti-inflammatory activities. Oral antibiotics do not typically produce satisfactory lesion clearance. In general, oral antibiotics used in the treatment of acne are slow-acting and require a treatment period of 3-6 months for optimum results. Hence compliance may be difficult, especially among younger patients. Long-term use of antibiotics is also associated with the spectre of bacterial antibiotic-resistance. Light-based therapies, such as 420-nm blue light or 1450-nm thermal lasers, can be used to treat mild to moderate acne based on their respective anti-bacterial photodynamic or thermal effect on sebaceous glands.

With current guidelines, the treatment regimen of choice for individuals with moderate to severe acne is oral antibiotics in combination with a dermatological agent such as a retinoid. For patients with recalcitrant nodular acne, first line therapy may consist of an oral retinoid, such as Accutane® (13-cis-retinoic acid). Accutane® has a strong inhibitory action on sebaceous glands and is therefore useful in removing comedones, reducing inflammation and inhibiting proliferation, differentiation and lipogenesis within sebaceous glands. In addition, Accutane® is also used to treat moderate or severe acne in patients at risk of physical or psychological scarring. Accutane® has long history of proven efficacy in treating acne. The majority of individuals treated with Accutane® experience remission with 3-6 months of daily dosing. In some cases, the treatment produces long-lasting benefit and is potentially curative. On the other hand, Accutane® is a recognized teratogen and is known to produce significant systemic adverse effects including elevated risk of mental depression, increased blood lipid levels and deleterious mucocutaneous changes. The strong inhibitory action of Accutane® on sebaceous gland activity clearly distinguishes it from the effects of dermatological retinoids and dermatological/oral antibiotics. However, topical treatment of acne is still preferred since this approach minimizes the risk of deleterious systemic effects associated with Accutane®. Drugs like Accutane®, which are effective orally, may have substantially less activity when administered topically, potentially due to their limited penetration into the skin and/or sebaceous glands.

Reducing sebum production as a means to treat acne has also been described. See, e.g., Zouboulis, C. C. et al., “Zileuton, an oral 5-lipoxygenase inhibitor, directly reduces sebum production”, Dermatology (2005), Vol. 210, pp. 36-38; and Zouboulis, C. C. et al., “A new concept for acne therapy: a pilot study with zileuton, an oral 5-lipoxygenase inhibitor, Arch. Dermatol. (2003), Vol. 139, pp. 668-670. Zileuton, an orally active inhibitor of 5-lipoxygenase, the enzyme that catalyzes the formation of leukotriene B4 (LTB4) from arachidonic acid, was tested on moderate to severe acne patients. LTB4 promotes production of sebum lipids. The results of this study revealed a 65% reduction of sebum lipids and a 71% reduction in inflammatory lesions at 12 weeks. This work indicated that acne could significantly improve with a non-retinoid that acts by inhibiting sebum production.

There exists a need, therefore, for a fast-acting, effective and safe dermatological or oral therapy for acne and other dermatological disorders which are characterized by sebaceous gland hyperactivity.

### SUMMARY OF THE INVENTION

Described herein are methods of using 5-(tetradecyloxy)-2-furancarboxylic acid (TOFA) and pharmaceutical compositions thereof for the treatment of dermatological disorders or conditions characterized by sebaceous gland hyperactivity, such as acne vulgaris, acne conglobata, choracne, rosacea, Rhinophyma-type rosacea, seborrhea, seborrheic dermatitis, sebaceous gland hyperplasia, Meibomian gland dysfunction of facial rosacea, mitogenic alopecia, and oily skin.

Accordingly, in one aspect, this invention is directed to a method of treating a human having a dermatological disorder or condition characterized by sebaceous gland hyperactivity, wherein the method comprises administering to the human in need thereof a therapeutically effective amount of 5-(tetradecyloxy)-2-furancarboxylic acid, or a pharmaceutically acceptable salt thereof.

One embodiment of this aspect is a method wherein the dermatological disorder or condition is selected from the group consisting of acne vulgaris, acne conglobata, choracne, rosacea, Rhinophyma-type rosacea, seborrhea, seborrheic dermatitis, sebaceous gland hyperplasia, Meibomian gland dysfunction of facial rosacea, mitogenic alopecia, and oily skin.

Of this embodiment, a preferred embodiment is a method wherein the dermatological disorder is acne.

Of this embodiment, another preferred embodiment is a method wherein the dermatological disorder is oily skin.

Another embodiment of this aspect is wherein the therapeutically effective amount of 5-(tetradecyloxy)-2-furancarboxylic acid, or a pharmaceutically acceptable salt thereof, is administered topically.

Another embodiment of this aspect is wherein the therapeutically effective amount of 5-(tetradecyloxy)-2-furancarboxylic acid, or a pharmaceutically acceptable salt thereof, is administered systemically.

Of this embodiment, a preferred embodiment is wherein the therapeutically effective amount of 5-(tetradecyloxy)-2-furancarboxylic acid, or a pharmaceutically acceptable salt thereof, is administered orally.

In another aspect, this invention is directed to a method of treating a human having a dermatological disorder or condition characterized by sebaceous gland hyperactivity, wherein the method comprises administering to the human in need thereof a pharmaceutical composition comprising a therapeutically effective amount of 5-(tetradecyloxy)-2-furancarboxylic acid, or a pharmaceutically acceptable salt thereof, and a pharmaceutically acceptable excipient.

One embodiment of this aspect is a method wherein the dermatological disorder or condition is selected from the group consisting of acne vulgaris, acne conglobata, choracne, rosacea, Rhinophyma-type rosacea, seborrhea, seborrheic dermatitis, sebaceous gland hyperplasia, Meibomian gland dysfunction of facial rosacea, mitogenic alopecia, and oily skin.

Of this embodiment, a preferred embodiment is wherein the dermatological disorder is acne.

Of this embodiment, another preferred embodiment is wherein the dermatological condition is oily skin.

Another embodiment of this aspect is wherein the therapeutically effective amount of 5-(tetradecyloxy)-2-furancarboxylic acid, or a pharmaceutically acceptable salt thereof, is administered topically.

Another embodiment of this aspect is wherein the therapeutically effective amount of 5-(tetradecyloxy)-2-furancarboxylic acid, or a pharmaceutically acceptable salt thereof, is administered systemically.

Of this embodiment, a preferred embodiment is wherein the therapeutically effective amount of 5-(tetradecyloxy)-2-furancarboxylic acid, or a pharmaceutically acceptable salt thereof, is administered orally.

Another embodiment of this aspect is wherein the pharmaceutical composition is a dermatological composition and the pharmaceutically acceptable excipient is a dermatologically acceptable excipient.

Another embodiment of this aspect is wherein the pharmaceutical composition is a systemic composition.

Of this embodiment, a more preferred embodiment is wherein the pharmaceutical composition is an oral composition.

Another aspect of this invention is directed to a method of inhibiting sebaceous gland activity in a human, wherein the method comprises administering to the human in need thereof a therapeutically effective amount of 5-(tetradecyloxy)-2-furancarboxylic acid, or a pharmaceutically acceptable salt thereof.

One embodiment of this aspect is wherein the therapeutically effective amount is administered topically.

Another embodiment of this aspect is wherein the therapeutically effective amount is administered systemically.

Of this embodiment, a preferred embodiment is wherein the therapeutically effective amount is administered orally.

Another aspect of this invention is directed to a method of inhibiting sebaceous gland activity in a human, wherein the method comprises administering to the human in need thereof a pharmaceutical composition comprising a therapeutically effective amount of 5-(tetradecyloxy)-2-furancarboxylic acid, or a pharmaceutically acceptable salt thereof, and a pharmaceutically acceptable excipient.

One embodiment of this aspect is wherein the therapeutically effective amount of 5-(tetradecyloxy)-2-furancarboxylic acid, or a pharmaceutically acceptable salt thereof, is administered topically.

Another embodiment of this aspect is wherein the therapeutically effective amount of 5-(tetradecyloxy)-2-furancarboxylic acid, or a pharmaceutically acceptable salt thereof, is administered systemically.

Of this embodiment, a preferred embodiment is wherein the therapeutically effective amount of 5-(tetradecyloxy)-2-furancarboxylic acid, or a pharmaceutically acceptable salt thereof, is administered orally.

Another embodiment of this aspect is wherein the pharmaceutical composition is a dermatological composition and the pharmaceutically acceptable excipient is a dermatologically acceptable excipient.

Another embodiment of this aspect is wherein the pharmaceutical composition is a systemic composition.

Of this embodiment, a preferred embodiment s wherein the pharmaceutical composition is an oral composition.

Another aspect of this invention is directed to pharmaceutical composition comprising a therapeutically effective amount of 5-(tetradecyloxy)-2-furancarboxylic acid or a pharmaceutically acceptable salt thereof, and a pharmaceutically acceptable excipient.

One embodiment of this aspect is wherein the pharmaceutical composition is a dermatological composition comprising a dermatologically effective amount of 5-(tetradecyloxy)-2-furancarboxylic acid or a pharmaceutically acceptable salt thereof, and a dermatologically acceptable excipient.

Of this embodiment, a preferred embodiment is wherein the dermatological composition is a gel formulation, an alcoholic gel formulation, a hydroalcoholic gel formulation, or a cream formulation.

Another embodiment of this aspect is wherein the pharmaceutical composition is an oral composition comprising a dermatologically effective amount of 5-(tetradecyloxy)-2-furancarboxylic acid or a pharmaceutically acceptable salt, and a pharmaceutically acceptable excipient.

Another aspect of this invention is directed to a method of treating a human having a disorder or condition characterized by inflammation, wherein the method comprises administering to the human in need thereof a pharmaceutical composition comprising a therapeutically effective amount of 5-(tetradecyloxy)-2-furancarboxylic acid, or a pharmaceutically acceptable salt thereof, and a pharmaceutically acceptable excipient.

One embodiment of this aspect is wherein the disorder or condition is inflammatory acne.

Another embodiment of this aspect is wherein the therapeutically effective amount of 5-(tetradecyloxy)-2-furancarboxylic acid, or a pharmaceutically acceptable salt thereof, is administered topically.

Another embodiment of this aspect is wherein the therapeutically effective amount of 5-(tetradecyloxy)-2-furancarboxylic acid, or a pharmaceutically acceptable salt thereof, is administered systemically.

Of this embodiment, a preferred embodiment is wherein the therapeutically effective amount of 5-(tetradecyloxy)-2-furancarboxylic acid, or a pharmaceutically acceptable salt thereof, is administered orally.

Another embodiment of this aspect is wherein the pharmaceutical composition is a dermatological composition and the pharmaceutically acceptable excipient is a dermatologically acceptable excipient.

Another embodiment of this aspect is wherein the pharmaceutical composition is a systemic composition.

Of this embodiment, a preferred embodiment is wherein the pharmaceutical composition is an oral composition.

Another aspect of this invention is directed to a method of reducing T cell proliferation and cytokine secretion in a human having a disorder or condition characterized by inflammation, the method comprising administering to the human in need thereof a pharmaceutical composition comprising a therapeutically effective amount of 5-(tetradecyloxy)-2-furancarboxylic acid, or a pharmaceutically acceptable salt thereof, and a pharmaceutically acceptable excipient.

One embodiment of this aspect is wherein the disorder or condition is inflammatory acne.

Another embodiment of this aspect is wherein the therapeutically effective amount of 5-(tetradecyloxy)-2-furancarboxylic acid, or a pharmaceutically acceptable salt thereof, is administered topically.

Another embodiment of this aspect is wherein the therapeutically effective amount of 5-(tetradecyloxy)-2-furancarboxylic acid, or a pharmaceutically acceptable salt thereof, is administered systemically.

Of this embodiment, a preferred embodiment is wherein the therapeutically effective amount of 5-(tetradecyloxy)-2-furancarboxylic acid, or a pharmaceutically acceptable salt thereof, is administered orally.

Another embodiment of this aspect is wherein the pharmaceutical composition is a dermatological composition and the pharmaceutically acceptable excipient is a dermatologically acceptable excipient.

Another embodiment of this aspect is wherein the pharmaceutical composition is a systemic composition.

Of this embodiment, a preferred embodiment is wherein the pharmaceutical composition is an oral composition.

The above aspects of the invention and embodiments thereof are described in more detail below.

### BRIEF DESCRIPTION OF FIGURES

FIG. 1 illustrates the effect of TOFA on Nile Red fluorescence profile of arachidonic acid (AA)-stimulated SZ95 sebocytes. Cells were treated with AA (100 μM) for 48 hours in the presence of different concentrations of TOFA and then stained with the neutral lipid-specific dye Nile Red. Cells were visualized using fluorescence microscopy.

FIG. 2A is a graph illustrating the effect of TOFA on lipid production and viability of AA-stimulated SZ95 sebocytes. Cells were treated with AA (100 μM) for 72 hours in the presence of different concentrations of TOFA. Cell viability was determined by MTS assay while lipid levels were assessed by Nile Red staining. Mean±standard deviations are shown.

FIG. 2B is a graph illustrating the effect of TOFA on viability of AA-stimulated SZ95 sebocytes at higher concentrations than in FIG. 2A.

FIG. 3 is a graph illustrating the effect of higher concentrations of TOFA on increased cytotoxicity in AA-stimulated SZ95 cells. Cells were treated with different amounts of AA in the presence of a concentration range of TOFA. Cell viability was determined by MTS assays performed after a 72 hour incubation period. Medium and treatments were replaced after the first 48 hours.

FIG. 4 is a graph illustrating the effect of TOFA on lipid production and viability of AA-stimulated SZ95 sebocytes. Following a 48 hour conditioning period in medium, sebocytes were treated with AA (100 μM) for 48 hours in the presence of different concentrations of TOFA. Cell viability was determined by MTS assay while lipid levels were assessed by Nile Red staining.

FIG. 5 is a graph illustrating the effect of TOFA pre-conditioning on lipid production and viability of AA-stimulated SZ95 sebocytes. Following a 48 hours in the presence of TOFA, cells were treated with AA (100 μM) for 48 hours without or with TOFA. Cell viability was determined by MTS assays while lipid levels were assessed by Nile Red staining.

FIG. 6 is a graph illustrating the influence of combined AA and TOFA pre-conditioning on lipid production and viability of AA-stimulated SZ95 sebocytes. Following a 48 hour conditioning period in the presence of AA plus TOFA, cells were treated with AA (100 μM) for 48 hours in the presence or absence of TOFA. Cell viability was determined by MTS assay while lipid levels were assessed by Nile Red staining.

FIG. 7 is a graph illustrating the lipid levels of SZ95 sebocytes pre-stimulated with AA. Following 48 hours in the presence of AA, cells were the treated with different amounts of TOFA. AA was retained in the culture system. Lipid levels were assessed by Nile Red staining at increasing culture times.

FIG. 8 illustrates dose-dependent effects of TOFA on differentiation and lipid accumulation in 3T3-L1 cells. Cells were imaged by phase-contrast microscopy. In the control cultures, lipid is clearly evident as spherical refractive intracellular droplets.

FIG. 9 illustrates the effect of TOFA on adipocyte differentiation and lipid accumulation in 3T3-L1 cells. Different amounts of TOFA were added to the cells at different stages of the differentiation process. Culture plates were subsequently stained with Oil Red O to detect neutral lipid. Shown images were scanned.

FIG. 10 illustrates the effect of TOFA on adipocyte differentiation and lipid accumulation in 3T3-L1 cells. Different amounts of TOFA were added to the cells at different stages of the differentiation process. Culture plates were subsequently stained with Oil Red O to detect neutral lipid. Cells were visualized by microscopy.

FIG. 11 illustrates the effect of TOFA on lipid accumulation in DHT-treated LNCaP cells. Different amounts of TOFA were added in the presence of DHT and cells were cultured for 96 hours at 37° C. Culture plates were subsequently stained with Oil Red O to detect neutral lipid and cells were visualized by microscopy.

FIG. 12 is a graph illustrating the inhibition of lipid synthesis in DHT-stimulated LNCaP cells by TOFA. Cells were cultured for 96 hours in the presence of DHT and different amounts of TOFA. Lipid accumulation was subsequently quantified by Nile Red staining and flow cytometric analyses.

FIG. 13 is a graph illustrating the inhibition of the increase in supernatant cytokine levels for PHA-stimulated PBMC by TOFA. Culture supernatants were obtained 48 hours following treatment and evaluated for cytokine levels using a flow-cytometry-based assay system. Results are expressed as a percentage (%) of the supernatant cytokine concentration determined for PBMC stimulated with PHA in the absence of TOFA.

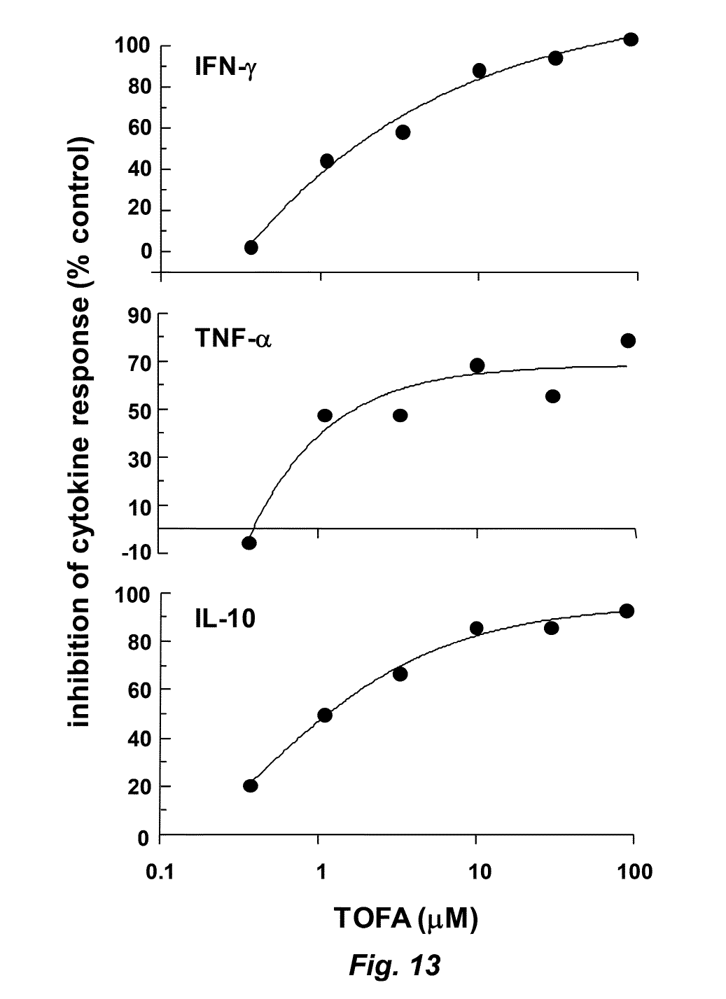

### DETAILED DESCRIPTION OF THE INVENTION

### Definitions

As used in the specification and appended claims, unless specified to the contrary, the following terms have the meaning indicated:

“Dermatological disorder or conditions” includes disorders involving hyperactive sebaceous gland activity including, for example, acne vulgaris, acne conglobata, choracne, rosacea, Rhinophyma-type rosacea, seborrhea, seborrheic dermatitis, sebaceous gland hyperplasia, Meibomian gland dysfunction of facial rosacea, mitogenic alopecia, and oily skin.

“Dermatologically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier, including those approved by the United States Food and Drug Administration as being acceptable for dermatological use on humans or domestic animals, or which are known, or are suitable for use in dermatological compositions.

“Dermatologically effective amount” refers to that amount of an active ingredient which, when administered dermatologically (i.e., systemically or locally, including, for example, topically, intradermally, intravenously, orally or by use of an implant, that afford administration to the sebaceous glands) to a human, is sufficient to effect the desired treatment, as defined below, of the disorder or condition of interest in the human. The amount of an active ingredient which constitutes a “dermatologically effective amount” will vary depending on the active ingredient, the disorder or condition and its severity, and the age of the human to be treated, but can be determined routinely by one of ordinary skill in the art having regard to his own knowledge and to this disclosure.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for pharmaceutical use or are suitable for use in humans or domestic animals.

“Therapeutically effective amount” refers to that amount of an active ingredient which, when administered orally to a human, is sufficient to effect the desired treatment, as defined below, of the disorder or condition of interest in the human. The amount of an active ingredient which constitutes a “therapeutically effective amount” will vary depending on the active ingredient, the disorder or condition and its severity, and the age of the human to be treated, but can be determined routinely by one of ordinary skill in the art having regard to his own knowledge and to this disclosure.

“Treating” or “treatment” as used herein covers the treatment of the disorder or condition of interest in a human having the disorder or condition of interest, and includes:

(i) preventing the disorder or condition from occurring in the human, in particular, when such human is predisposed to the disorder or condition but has not yet been diagnosed as having the disorder or condition or;

(ii) inhibiting the disorder or condition, i.e., arresting its development;

(iii) relieving the disorder or condition, i.e., causing regression of the condition; or

(iv) stabilizing the disorder or condition.

“Pharmaceutically acceptable salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

### Utility of the Invention

Increased sebum production due to sebaceous gland hyperactivity is one of several factors generally believed to be contributors to acne pathogenesis. In the formation of sebum, there is stepwise differentiation of sebocytes, a specialized epithelial cell type, arising from basal progenitor cells leading to lipid-forming cells which as they progress toward the gland outlet. These enlarged cells ultimately rupture (holocrine secretion) releasing their lipid-rich content (sebum). The overall makeup of sebum consists of squalene (12%), cholesterol (2%), wax esters (26%), and diglycerides/triglycerides/free fatty acids (57%) (see, Zouboulis et al., “An oral 5-lipoxygenase inhibitor, directly reduces sebum production”. Dermatology. (2005) 210:36-38). Free fatty acid levels may be increased by bacterial degradation of the di- and triglycerides present within sebum (see, Thiboutot D. “Regulation of human sebaceous glands” J. Invest Dermatol. (2004) 123:1-12).

Free fatty acids may also promote the inflammatory aspects of acne by activating local immune cells and their release of a variety of pro-inflammatory factors.

Fatty acid synthesis starts with the carboxylation of acetyl CoA to malonyl CoA. This irreversible reaction is the committed step in fatty acid synthesis. The synthesis of malonyl CoA is catalyzed by acetyl CoA carboxylase (ACC) (See, Brownsey, R. W. et al., “Regulation of acetyl-CoA carboxylase”, Biochem Soc. Trans. (2006) 34: 223-227). ACC exists as two tissue-specific isoforms, a single-chain 265 kDa protein (ACC1), and a 280 kDa protein (ACC2) (See, Waldrop, G. L. et al., “Targeting acetyl-CoA carboxylase for anti-obesity therap,” Curr. Med. Chem.—Immun., Endoc. & Metab. Agents (2002) 3: 229-234).

In mammalian cells, ACC1 is present within the cytosol while ACC2 localizes to mitochondria. Generally, ACC1 is responsible for long-chain fatty acid synthesis while mitochondrial ACC2 acts to inhibit fatty acid oxidation. Expression of the ACC isoforms is tissue-specific and responsive to hormones and nutritional status. ACC1 is expressed at high levels in lipogenic tissues, notably in adipose, liver, and lactating mammary gland. ACC2 is a minor component of hepatic ACC and is the predominant isoform expressed, albeit at relatively low levels, in heart and skeletal muscle. Active ACC has been shown to be present in human sebaceous glands, although the ACC isoform expression pattern has not yet been described (see, Smythe, C. D. et al., “The activity of HMG-CoA reductase and acetyl-CoA carboxylase in human apocrine sweat glands, sebaceous glands, and hair follicles is regulated by phosphorylation and by exogenous cholesterol, ” J. Invest. Dermatol. (1998) 111:139-148). ACC and other fatty acid and cholesterol synthesis-regulating enzymes have been shown to be positively regulated by androgen, a key factor contributing to the increased sebum production at puberty as well as the expression of acne (see, Rosignoli, C. et al., “Involvement of the SREBP pathway in the mode of action of androgens in sebaceous glands in vivo”, Exp. Dermatol. (2003) 12:480-489).

ACC also catalyzes the first committed and regulated step in fatty acid synthesis in bacteria. Since membrane lipid biogenesis is essential for bacterial growth, inhibition of ACC activity may potentially decrease the growth of bacteria normally present within a comedone.

Long-chain (16-20 carbons) fatty acid acyl-CoA thioesters have been found to be potent physiological end-product inhibitors of mammalian ACC.

TOFA (5-(tetradecyloxy)-2-furancarboxylic acid) is a known hypolipidemic compound having the following structure:

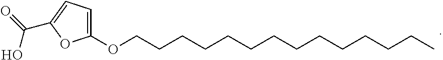 [SMILES: CCCCCCCCCCCCCCOc1ccc(C(=O)O)o1]

TOFA, or a pharmaceutically acceptable salt thereof, is described and claimed in U.S. Pat. No. 4,110,351 (the disclosure of which is incorporated in full by reference) and is commercially available, for example, from Cedarlane Laboratories, Inc. TOFA has been shown to reduce plasma triglyceride levels in both rats and monkeys (see, e.g., Parker, R. A. et al., J. Med. Chem. (1977), Vol. 20, pp. 781-791) and to inhibit hepatic fatty acid synthesis (see, e.g., Ribereau-Gayon, G., FEBS Lett. (1976), Vol. 62, No. 309-312; Panek, E. et al., Lipids (1977), Vol. 12, pp. 814-818; Kariya, T. et al., Biochem. Biophys. Res. Commun. (1978), Vol. 80, pp. 1022-1024; and Harris, R. A. et al., Hormones and Energy Metabolism (Klachko, D. M. et al., eds.), Vol. III, pp. 17-42.

TOFA, when converted intracellularly to its acyl-CoA thioester, inhibits ACC activity with a mechanism similar to long chain fatty acyl-CoA's, the physiological end-product inhibitors of ACC (see, McCune, S. A. et al., J. Biol. Chem. (1979), Vol. 254, No. 20., pp. 10095-10101. As a fatty acid mimetic, TOFA may exert multiple effects in sebaceous gland disorders by lowering sebum production and potentially affecting the growth of pathogenic bacteria at the treatment site.

Accordingly, and without wishing to be bound by theory, this invention is directed to the use of TOFA, or pharmaceutical or dermatological compositions thereof, to treat dermatological disorders or conditions characterized by sebaceous gland hyperactivity, such as acne and oily skin, through the inhibition of ACC activity in sebocytes.

### Testing of the Invention

Study of human sebocyte function has been relatively restricted due to the lack of suitable cell lines. Recently, SZ95 sebocytes were prepared using human facial sebaceous gland cells transfected with a plasmid containing the coding region for the Simian virus-40 large T antigen (see, Zouboulis, C. C. et al., J. Invest. Dermatol. (1999), Vol. 113, pp. 1011-1020). SZ95 cells express a number of molecules typically associated with human sebocytes. Functional studies showed synthesis of the sebaceous lipids squalene and wax esters as well as triglycerides and free fatty acids (see, Zouboulis C C, Seltmann H, Neitzel H, Orfanos C E. Establishment and characterization of an immortalized human sebaceous gland cell line (SZ95). J. Invest Dermatol. (1999) 113:1011-1020).

Thus, SZ95 cells are capable of recapitulating many aspects of sebocyte growth and differentiation (see, Wrobel, A. et al., “Differentiation and apoptosis in human immortalized sebocytes”, J. Invest Dermatol. (2003) 120:175-181).

Treatment with arachidonic acid (AA) reproducibly increased SZ95 sebocyte lipid levels approximately 5-fold using a 96-well microtiter plate format. SZ95 cells can be used to identify compounds with sebum-inhibitory potential, such as Accutane® and cholesterol synthesis inhibitors (statins), both of which demonstrated the ability to lower lipid production by these cells (See, Tsukada, M. et al., “13-cis retinoic acid exerts its specific activity on human sebocytes through selective intracellular isomerization to all-trans retinoic acid and binding to retinoid acid receptors”, J. Invest. Dermatol. (2000) 115:321-327).

As demonstrated in more detail below in Examples 1-3, the administration of TOFA to SZ95 cells resulted in lower lipid production in these cells, as well as other lipid-producing cells. Accordingly, TOFA would be a useful agent in treating disorders or conditions characterized by hyperactivity of sebaceous glands, particularly by an increased sebum production in sebocytes.

Also, as demonstrated in more detail below in Example 4, the administration of TOFA inhibited several parameters related to T cell activation including proliferation and secretion of immune/inflammation-regulating cytokines. Accordingly, TOFA would be a useful agent in treating dermatological disorders or conditions characterized by inflammation, by reducing T cell proliferation and cytokine secretion, for example, in the treatment of inflammatory acne.

### EXAMPLE 1

### Inhibition of Lipid Synthesis in SZ95 Sebocytes

### SZ95 Sebocyte Culture and Induction of Lipid Synthesis

The immortalized human sebocyte cell line, SZ95, was maintained in culture as described in Zouboulis, C. C. et al., J. Invest. Dermatol. (1999), Vol. 113, pp. 1011-1020. Lipid synthesis was stimulated by treating SZ95 cells with arachidonic acid (AA). For measurement of lipid production and lipid inhibition studies, test compounds were dissolved in dimethylsulfoxide (DMSO) and added at the desired concentration in 96-well microtiter plates. The cells were then cultured for up to 72 hours before the plates were washed 3 times with PBS and a final volume of 200 μL PBS/well was added. To stain cell neutral lipids, 5 μL of Nile Red solution (0.2 mg/mL dissolved in DMSO) was added to each well and incubated for a minimum of 60 minutes. Plate fluorescence was then quantified using a fluorometric plate reader (excitation wavelength: 490 nm; emission wavelength: 590 nm). Inhibition of lipid levels by the test compound was expressed as the % reduction of the fluorescence of AA-stimulated cells in the presence of the test compound relative to the values obtained for the unstimulated control cells. Cell viability was measured by utilizing the conversion of a tetrazolium reagent (MTS) to a colored-formazan product by live cells. For these assays, the test compound was dissolved in dimethylsulfoxide (DMSO) and added at the desired concentration to cells seeded into 96-well plates. The cells were cultured for 48 hours in the presence of the test compound before the plates were washed 3 times with PBS. A final volume of 100 μL of culture medium per well was added. Twenty μL of MTS solution (0.2 mg/mL in sterile PBS) was added to each well and incubated for a minimum of 60 minutes until the desired optical density was reached. The color development of the wells was measured using a plate reader at an absorbance of 590 nm. Effect on cell viability by the test compound was expressed as the % reduction of the absorbance for AA-stimulated cells in the presence of the test compound relative to the values obtained for the untreated control cells.

### Results

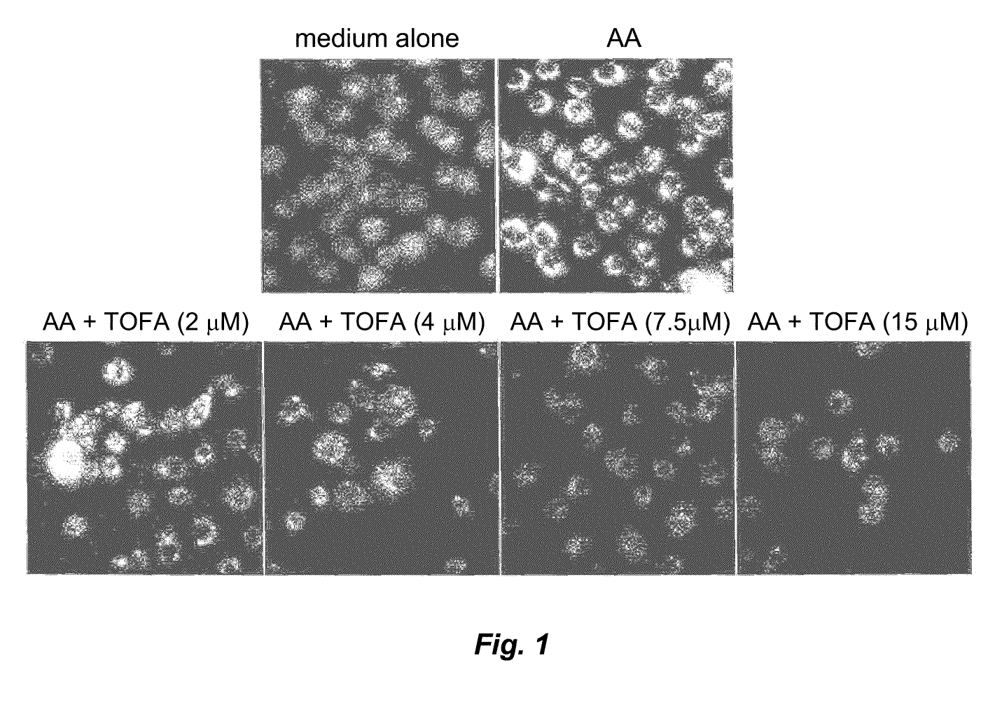

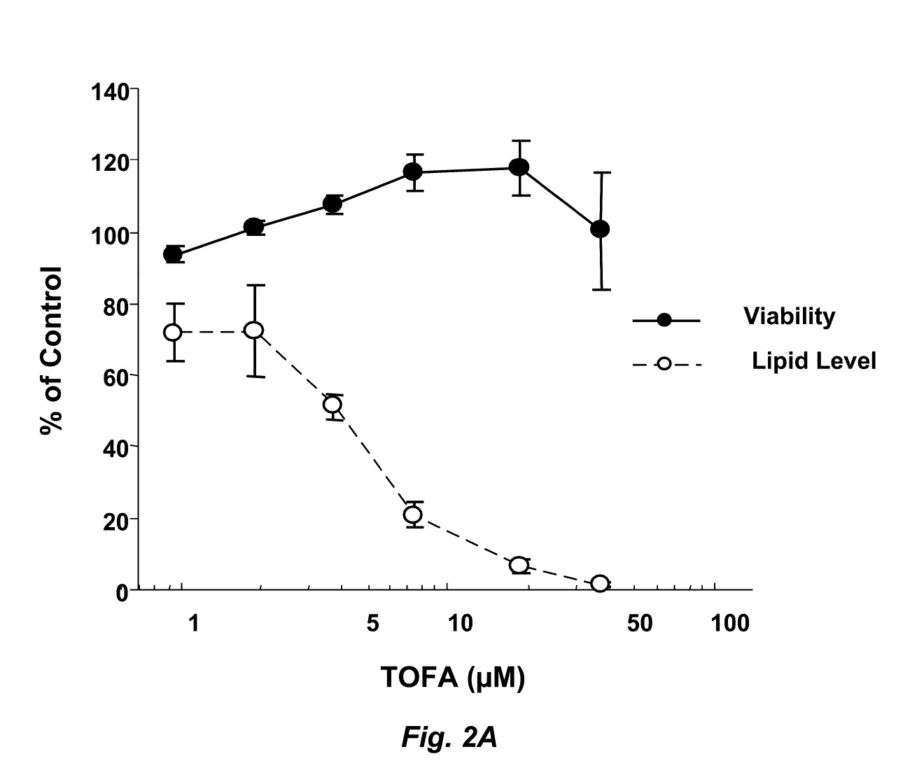

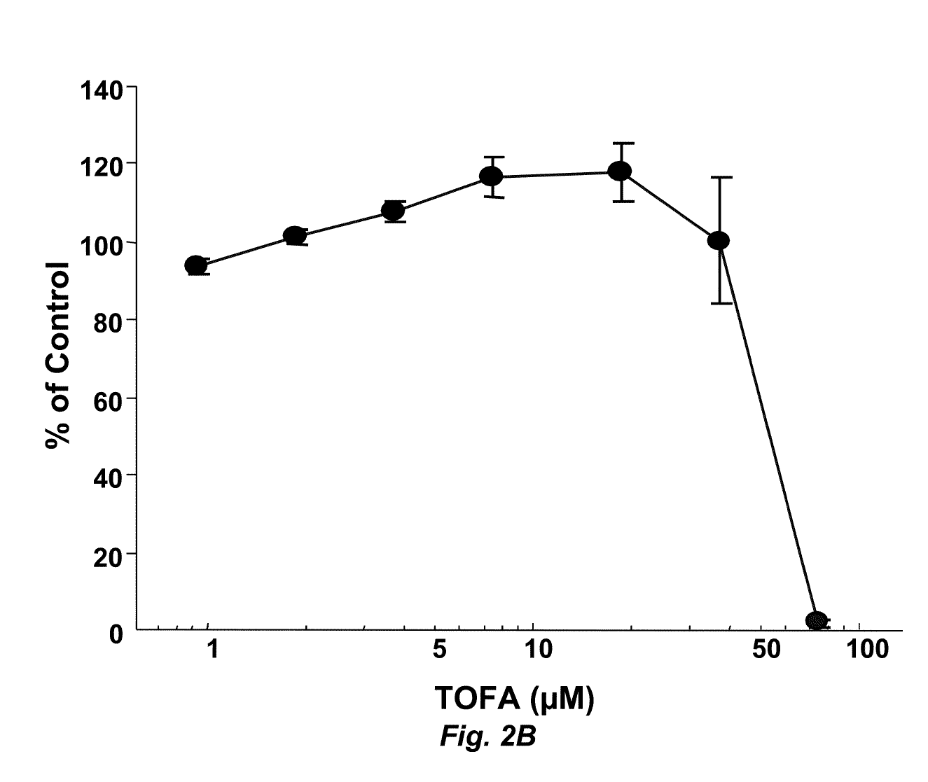

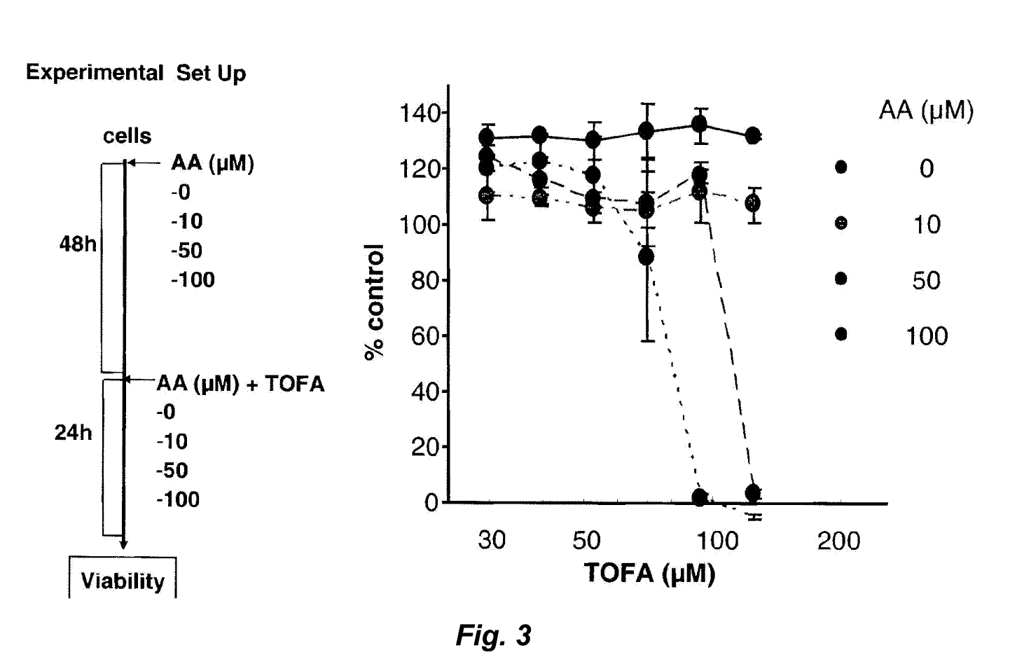

AA-stimulated SZ95 cells treated with TOFA showed a dose-dependent inhibition of lipid synthesis as observed by fluorescence microscopy (FIG. 1). With nile red fluorescence intensity measurements, the estimated IC₅₀ for TOFA upon lipid synthesis was ˜3 μM (FIG. 2A). In comparison, the IC₅₀ for TOFA against lipid synthesis by 13-cis retinoic acid in cells was ˜40 μM in short term cultures. Thus, TOFA was shown to be a relatively potent inhibitor of sebocyte lipid synthesis. AA-stimulated SZ95 cells exhibited viability loss at TOFA concentrations greater than 50 μM (FIG. 2B). The cytotoxic effect of TOFA was more pronounced when the cells were treated with higher AA concentrations (FIG. 3). However, pronounced inhibition of SZ95 sebocyte lipid formation was evident at TOFA concentrations that were non-cytotoxic.

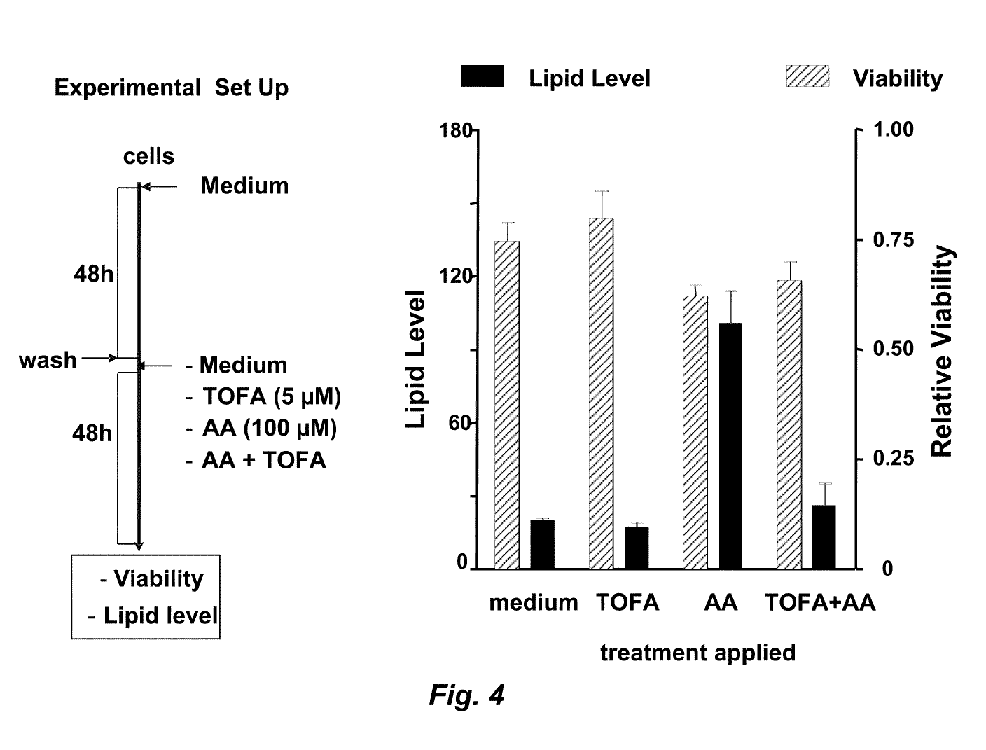

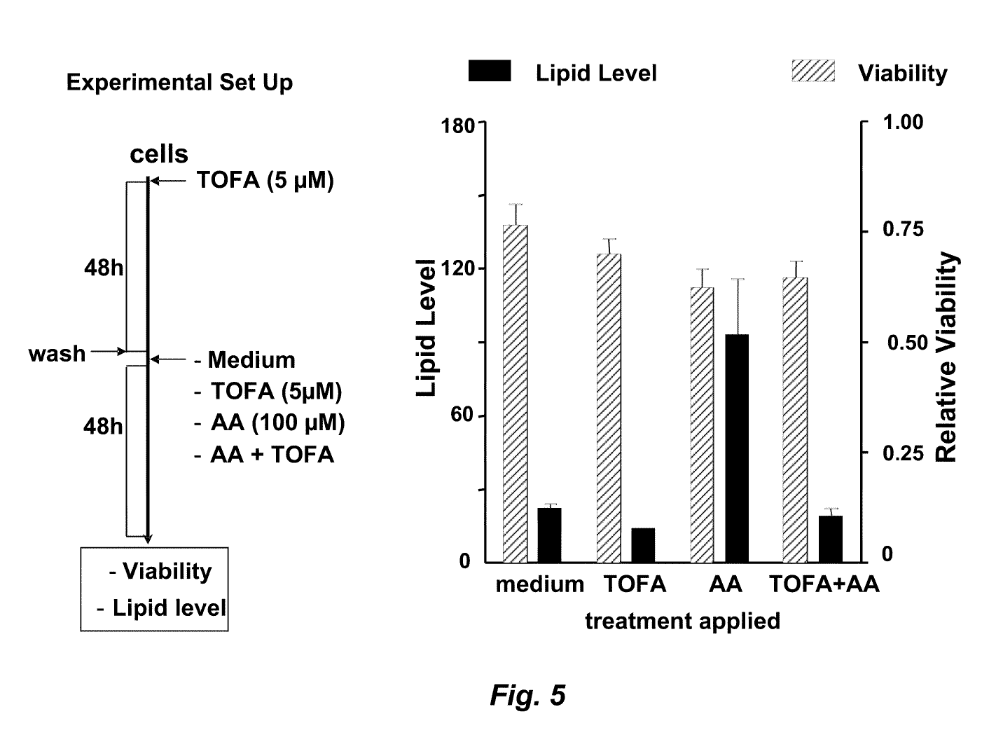

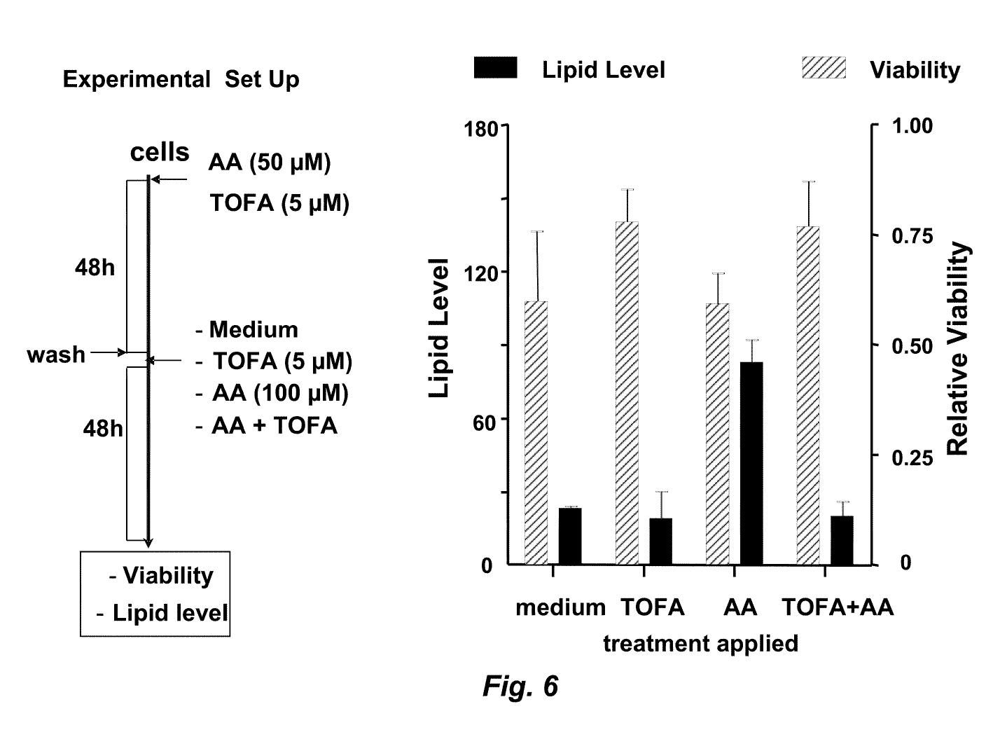

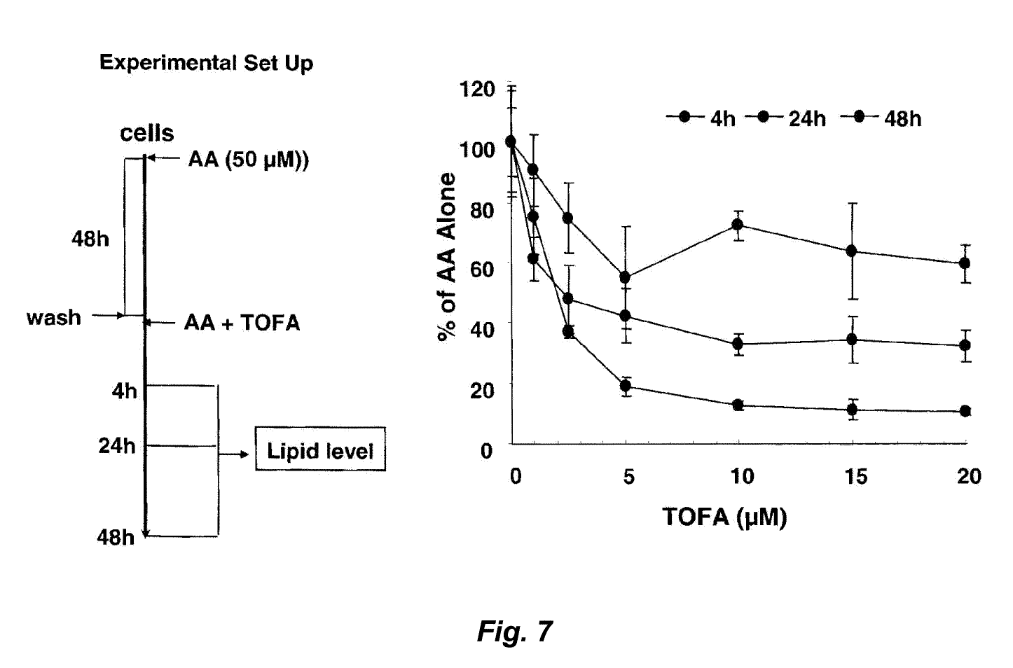

SZ95 sebocytes showed reduced lipid synthesis when TOFA was added simultaneously with AA stimulation (FIG. 4). While not wishing to be bound by any theory, FIG. 7 suggests that inhibition of new lipid synthesis, lipid oxidation and/or metabolism may be effected by TOFA, thereby effecting lipid levels. These cells also exhibited impaired lipid synthesis when TOFA was added 48 hours prior to AA stimulation (FIG. 5). Removal of TOFA prior to AA stimulation resulted in lipid levels that approach control levels. Further, removal of TOFA in the setting of continued AA stimulation resulted in restoration of lipid levels to near normal levels (FIG. 6). These findings indicated the reversible nature of ACC inhibition with TOFA.

SZ95 cells also demonstrated reduced lipid levels when TOFA was added following 48 hours of AA pre-stimulation (FIG. 7). Furthermore, when lipid levels were measured following different TOFA treatment times, accumulated lipid levels were reduced in both a time and dose dependent pattern.

### EXAMPLE 2

### Effect of TOFA on Lipid Accumulation by LNCaP Cells

### Culture and Stimulation of Lipid Droplet Formation in LNCaP Cells

The human prostate LNCaP adenocarcinoma cell line was obtained from American Type Culture Collection. Cells were maintained in RPMI 1640 medium containing 10% fetal calf serum (FCS), 4 mM Glutamax, 1 mM sodium pyruvate, 1 mM HEPES, penicillin (100 U/mL) and streptomycin (100 μg/mL). For experiments, approximately 10,000 cells/well were plated in 6-well tissue culture plates in RPMI 1640 10% FBS for 72 hours. To minimize potential serum androgen effects, medium containing 5% charcoal/dextran-stripped FCS was added for 72 hours. Lipid synthesis was then stimulated by addition of the androgen dihydrotestosterone (DHT) at 50 nM. TOFA was solubilized in DMSO and added at various concentrations in RPMI 1640 containing 5% charcoal/dextran-treated. Cells were incubated in the presence of these factors for 96 hours at 37° C. Lipid accumulation was subsequently quantified by Nile Red staining and flow cytometric analysis. The flow cytometer laser excitation was set at 488 nm and Nile Red fluorescence collected through a 585 nm band pass filter. A total of 10,000 events were collected per sample. The lipid level of test compound-treated wells was compared to the result obtained for the vehicle-treated cells. Cell Nile Red fluorescence was also visualized through a fluorescence microscope.

Cellular lipid expression was also determined using Oil Red O staining. Cells were fixed in 10% formalin for 15 minutes and stained with Oil Red O working solution (0.18% Oil-Red-O dye/60% propanol) for 2 hours. Plates or dishes were rinsed in distilled water until the desired color development was achieved in the control wells. Oil Red O-stained images were captured. Lipid accumulation was visualized by fluorescence microscopy.

### Results

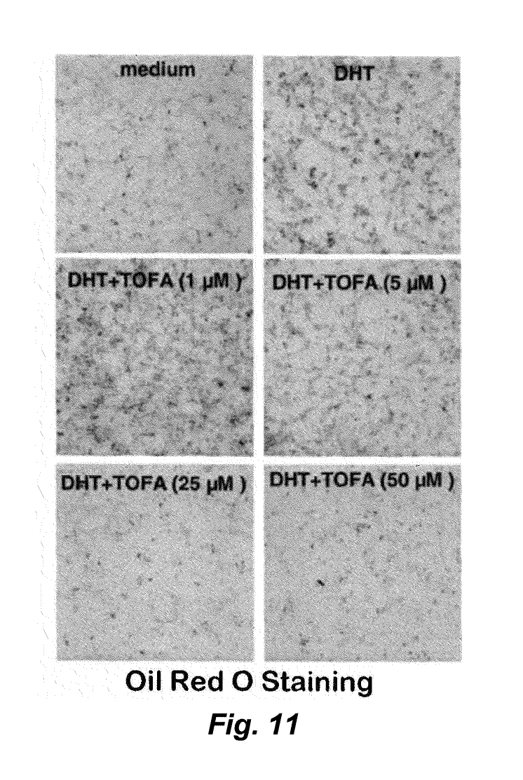

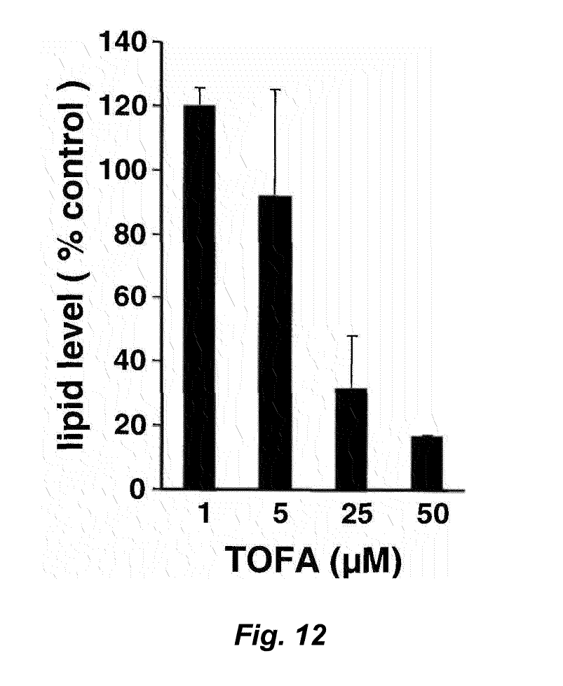

For DHT-stimulated LNCaP cells, TOFA produced a dose dependent reduction in lipid levels as indicated by Oil Red O staining and fluorescence microscopy (FIG. 11). Quantification of lipid levels using a novel flow cytometry/fluorescence method revealed>50% reduction produced by TOFA at 25 μM (FIG. 12).

### EXAMPLE 3

### Effect of TOFA on 3T3-L1 Adipocyte Differentiation and Lipid Accumulation

### Culture and Adipocyte Differentiation of 3T3-L1 Cells

Mouse 3T3-L1 preadipocytes (American Type Culture Collection) were passaged and maintained in Dulbecco's modified Eagles Medium (DMEM) supplemented with 10% fetal calf serum (FCS), 1 mM sodium pyruvate, penicillin (100 U/ml)/streptomycin (100 μg/ml) and 4 mM Glutamax (Gibco/Life Technologies). To initiate adipocyte differentiation, 3T3-L1 cells were plated at confluency into culture plates or dishes and grown in supplemented DMEM for two days post-confluency. Initiation medium consisted of DMEM with 0.5 mM 3-isobutyl-1-methylxanthine, 1 μM dexamethasone and human insulin at 10 μg/ml. Progression medium contained insulin (10 μg/ml) which replaced the initiation medium after 48-72 hours. Cellular lipid was imaged by Oil Red O staining as described in Example 2 for LNCaP cells.

### Results

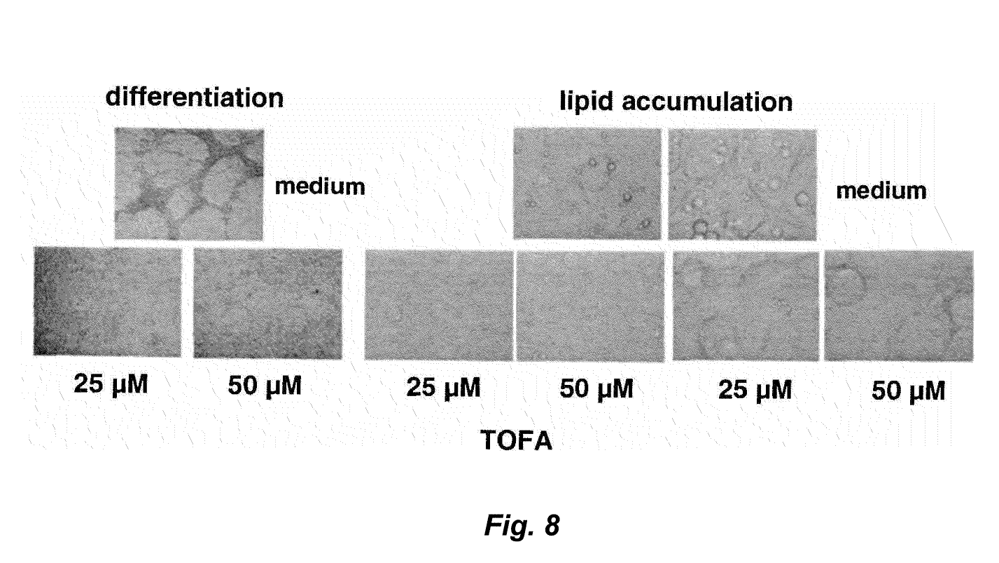

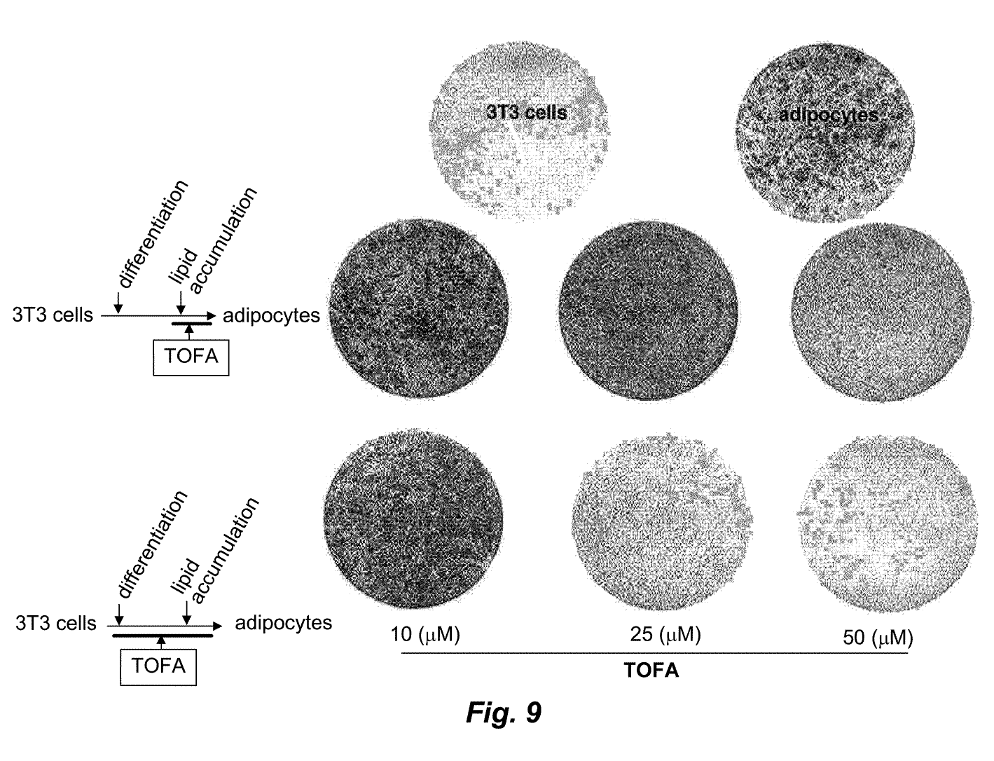

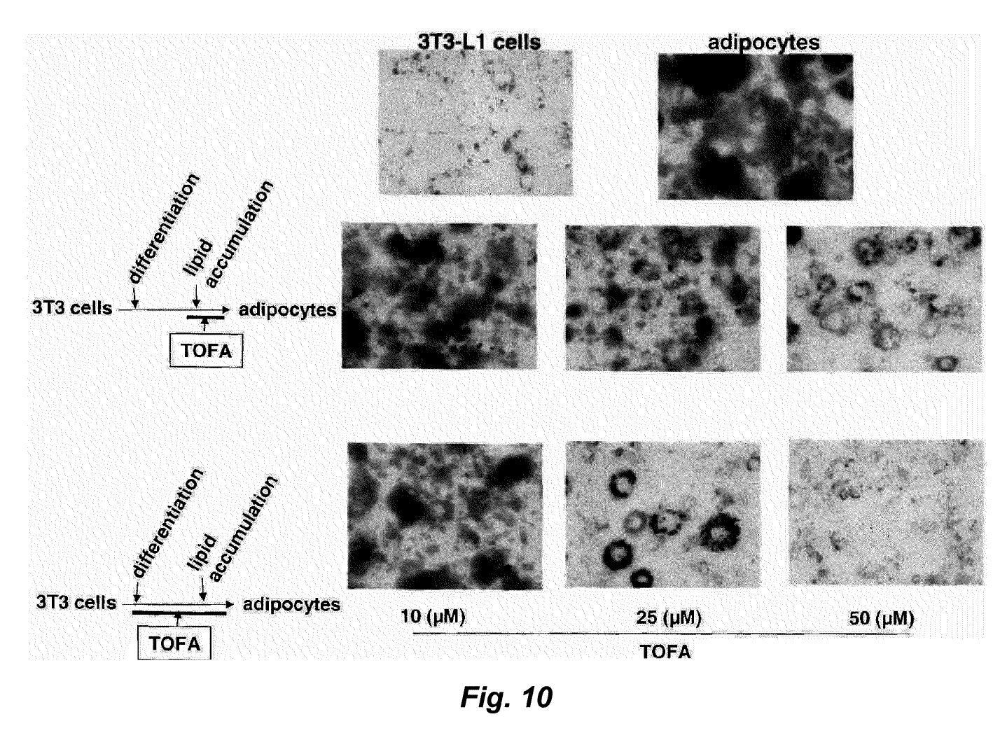

For mouse 3T3-L1 adipocytes, TOFA exerted dose-dependent effects on cellular differentiation and lipid accumulation (FIGS. 8-10). Furthermore, TOFA had a reductive effect on lipid accumulation when added to cells that had undergone adipocyte differentiation (FIGS. 9-10).

Overall, as above Examples 1-3 demonstrate, TOFA significantly reduced lipid levels in three different cell types (SZ95 sebocytes, 3T3-L1 adipocytes, LNCaP prostate cancer cells) in vitro. Each cell system utilizes distinct stimuli to up-regulate lipid synthesis. The ability of TOFA to impact lipid synthesis within these different experimental settings supports the premise that this compound affects a lipid biosynthetic pathway component common to all cell types.

### EXAMPLE 4

### Effect of TOFA on Proliferation and Cytokine Production by Activated Human Peripheral Blood Mononuclear Cells (PBMC)

As described herein, the inhibitory effect of TOFA on fatty acid synthesis is well known. However, the potential influence of TOFA on cellular production of inflammatory mediators or immune cell proliferation is unreported. To address this question, PBMC were isolated from different donors by density gradient centrifugation. Different amounts of TOFA were added to PBMC cultures in the presence of two different stimuli sets. One activating stimulus was phytohemagglutinin (PHA), a plant-derived mitogen that stimulates proliferation and cytokine synthesis by T lymphocytes. These cell preparations were also activated using a combination of interferon-γ (IFN-γ) and lipopolysaccharide (LPS) to stimulate cytokine production by the monocyte fraction within PBMC preparations. Following a 48 hour culture period, cell supernatants were obtained for simultaneous determination of cytokine levels using a flow cytometry-based quantification method. Cytokine levels were interpolated from a standard curve generated in parallel. Cell viability was assessed using a colorimetric assay based on the conversion of 3-(4,5-dimethylthiazol-2-yl)-5-(3-carboxymethoxyphenyl)-2-(4-sulfophenyl)-2H-tetrazolium, inner salt (MTS) into a soluble formazan product by mitochondrial dehydrogenase of viable cells. Cell proliferation was determined by adding ³H-thymidine to the cultures and determining its level of incorporation into DNA using scintillation counting.

Three independent experiments were performed using PBMC obtained from different adult donors. In Experiment #2, the cell yield was insufficient to perform the full assay panel. As shown in Table 1 below, TOFA had no effect on the viability of PBMC stimulated with PHA or IFN-γ plus LPS:

TABLE 1
Effect of TOFA on proliferation and cytokine production by PPMC
stimulated with PHA (2 μg/mL) or IFN-γ (33 ng/mL) plus LPS
(10 ng/mL) in the presence of 10% fetal bovine serum (FBS) for
48 hours. Results are given as the concentration of TOFA required to
lower the assay readout by 50% (i.e. inhibitory concentration: IC₅₀)

| Stimulus | |
|---|---|
| PHA | IFN-γ + LPS |

| Experiment | Viability | Proliferation | Viability | Proliferation |
|---|---|---|---|---|
| 1 | >90 | 3.4 | >90 | >90 |
| 2 | >90 | not done | >90 | not done |
| 3 | >90 | 7.4 | >90 | 44.3 |

For PBMC treated with IFN-y plus LPS, ³H-thymidine incorporation was either unaffected or reduced at relatively high concentrations of TOFA. In contrast, the proliferation of PHA-stimulated PBMC was significantly inhibited by low micro-molar levels of TOFA.

Culture supernatants for TOFA-treated PBMC were tested for levels of different immune/inflammation-modulating cytokines. As shown in Table 2 below, for PBMC treated with IFN-γ plus LPS, TOFA had little effect on supernatant levels of interleukin-1β (IL-1β), IL-6, IL-8 or tumor necrosis factor-α (TNF-α). TOFA had a modest inhibitory effect on PBMC secretion of IL-12 with a significant degree of inter-experimental variability evident.

As shown below in Table 3, for PBMC stimulated with the T cell mitogen PHA, TOFA had a significant effect on IFN-γ, TNF-α and IL-10 supernatant levels. The effect on IFN-γ expression was relatively consistent among the three experiments performed while there was greater inter-donor variability on the impact of TOFA on PBMC secretion of TNF-α and IL-10. The results of Experiment #3 showing the effect of TOFA on supernatant levels of IFN-γ, TNF-α and IL-10 for PHA-stimulated PBMC are presented graphically in FIG. 1. Overall, TOFA inhibited several parameters related to T cell activation including proliferation and secretion of immune/inflammation-regulating cytokines. These unanticipated findings suggest that TOFA could have anti-inflammatory properties by reducing T cell proliferation and cytokine secretion. It has been demonstrated that T cells are present in inflammatory acne lesions and these cells secrete abundant amounts of IFN-γ with appropriate stimulation (see, Mouser, P. E. et al., “Propionibacterium acnes-reactive T helper-1 cells in the skin of patients with acne vulgaris”, J. Invest. Dermatol. (2003) 121: 1226-1228).

TABLE 2
Effect of TOFA on supernatant cytokine levels for PBMC stimulated with
IFN-γ plus LPS in the presence of 10% FBS for 48 hours. Results are
given as the estimated concentration of TOFA required to lower
supernatant cytokine levels by 50% (IC₅₀).

| | Cytokine | | | | |
|---|---|---|---|---|---|
| Experiment | IL-1β | IL-6 | IL-8 | IL-12 | TNF-α |
| 1 | >90 | >90 | >90 | ~30 | >90 |
| 2 | >90 | >90 | >90 | 83.0 | >90 |
| 3 | >90 | >90 | >90 | 11.7 | >90 |

TABLE 3
Influence of TOFA on supernatant cytokine levels for PBMC stimulated
with PHA for 48 hours Results are given as the estimated concentration
of TOFA required to lower supernatant cytokine levels by 50% (IC₅₀).

| | Cytokine | | |
|---|---|---|---|
| Experiment | IFN-γ | TNF-α | IL-10 |
| 1 | 1.6 | 1.2 | 26.0 |
| 2 | 6.4 | 35.0 | 0.8 |
| 3 | 6.8 | 3.7 | 4.9 |

### Pharmaceutical Compositions of the Invention and Administration

Pharmaceutical compositions comprising TOFA, or a pharmaceutically acceptable salt thereof, and a pharmaceutically acceptable excipient are one aspect of the present invention. These pharmaceutical compositions may be in any form which allows for the active ingredient, i.e., TOFA, to be administered to a human in a therapeutically effective amount. For example, the pharmaceutical composition may be in the form of a semi-solid (gel), solid, liquid or gas (aerosol). Typical routes of administration include, without limitation, systemic (including oral and parenteral), topical, buccal, transdermal, sublingual, nasal, rectal, vaginal, and intranasal administration. The term parenteral as used herein includes subcutaneous injections, needle-less injections, intravenous, intramuscular, epidural, intrasternal injection or infusion techniques. Pharmaceutical compositions of the invention are formulated so as to allow the active ingredients contained therein to be bioavailable upon administration of the composition to a human. Pharmaceutical compositions of the invention that will be administered to a human may take the form of one or more dosage units, where for example, a tablet, capsule, cachet or patch may be a single dosage unit, and a container of a pharmaceutical composition of the invention in aerosol form may hold a plurality of dosage units.

In treating dermatological disorders characterized by sebaceous gland hyperactivity, TOFA is preferably administered to the skin (i.e., topically) of the human in need thereof in dermatologically acceptable compositions, as described in more detail below. When such compositions are in use (e.g., when a dermatological composition comprising TOFA and a dermatologically acceptable excipient is placed upon the skin of the human in need thereof), TOFA is in continuous contact with the skin of the patient, thereby effecting treatment.

Any suitable amount of TOFA can be employed in such dermatological compositions, provided the amount employed effectively inhibits the production of sebum from sebocytes and remains stable in the composition over a prolonged period of time. Preferably, the stability is over a prolonged period of time, e.g., up to about 3 years, up to 1 year, or up to about 6 months, which is typical in the manufacturing, packaging, shipping and/or storage of dermatologically acceptable compositions. TOFA can be solution, partially in solution with an undissolved portion or completely undissolved suspension. TOFA can be present in a dermatological composition of the invention in a concentration range from about 0.001 wt. % to about 80 wt. %, from about 0.001 wt. % to about 50 wt. %, from about 0.001 wt. % to about 25 wt. %, or from about 0.001 wt. % to about 6 wt. % of the dermatological composition. In one embodiment, TOFA can be present in a concentration range of from about 0.001 wt. % to about 10 wt. %, from about 0.1 wt. % to about 10 wt. % or from about 1.0 wt. % to about 5.0 wt. % of the dermatological composition. In another embodiment of the invention, a dermatological TOFA formulation to be administered topically contains (by weight) about 3% TOFA in about 40% dimethylacetamide (DMA)/30% acetone/30% ethanol.

A dermatological composition of the invention can be in the form of a solution, lotion, foam, gel, cream and/or ointment. Preferably, the dermatological composition will be a topical formulation, for example, a gel, foam, cream or ointment.

A dermatological composition of the invention can contain one or more “lipophilic solvent(s)” that acts as a carrier into the pilosebaceous unit. A lipophilic solvent useful in the invention can be miscible with water and/or lower chain alcohols and have a vapor pressure less than water at 25° C. (˜23.8 mm Hg). A lipophilic solvent useful in the invention can be a glycol, specifically propylene glycol. In particular, the propylene glycol can be from the class of polyethylene glycols, specifically polyethylene glycols ranging in molecular weight from 200 to 20000. Preferably, the solvent would be part of a class of glycol ethers. More specifically, a lipophilic solvent of the invention would be diethylene glycol monoethyl ether (transcutol). As used herein, “diethylene glycol monoethyl ether” (“DGME”) or “transcutol” refers to 2-(2-ethoxyethoxy)ethanol {CAS NO 001893} or ethyoxydiglycol.

A dermatological composition of the invention can also contain one or more “filler(s)” that has a vapor pressure greater than or equal to 23.8 mm Hg at 25° C. The filler should have a vapor pressure greater than or equal to the lipophilic solvent as to concentrate TOFA on the skin. Preferred concentration range of a single filler or the total of a combination of fillers can be from about 0.1 wt. % to about 10 wt. %, more preferably from about 10 wt. % to about 50 wt. %, more specifically from about 50 wt. % to about 95 wt. % of the dermatological composition. Non-limiting examples for use herein include water and lower alcohols, including ethanol, 2-propanol and n-propanol. More preferably, the filler is water, ethanol and/or 2-propanol. Specifically, the filler would be ethanol and/or water.

A dermatological composition of the invention can also contain one or more “humectant(s)” used to provide a moistening effect. Preferably the humectant remains stable in the composition. Any suitable concentration of a single humectant or a combination of humectants can be employed, provided that the resulting concentration provides the desired moistening effect. Typically, the suitable amount of humectant will depend upon the specific humectant or humectants employed. Preferred concentration range of a single humectant or the total of a combination of humectants can be from about 0.1 wt. % to about 70 wt. %, more preferably from about 5.0 wt. % to about 30 wt. %, more specifically from about 10 wt. % to about 25 wt. % of the dermatological composition. Non-limiting examples for use herein include glycerin, polyhydric alcohols and silicone oils. More preferably, the humectant is glycerin, propylene glycol and/or cyclomethicone. Specifically, the filler would be glycerine and/or cyclomethicone.

A dermatological composition of the invention can also contain a gelling agent that increases the viscosity of the final solution. The gelling agent can also act as an emulsifying agent. The present dermatogological compositions can form clear gels and soft gels, which upon application to the skin can break down and deteriorate, affording gels that do not dry on the skin. Typically, the concentration and combination of gelling agents will depend on the physical stability of the finished product. Preferred concentration range of a gelling agent can be from about 0.01 wt. % to about 20 wt. %, more preferably from about 0.1 wt. % to about 10 wt. %, more specifically from about 0.5 wt. % to about 5 wt. % of the dermatological composition. Non-limiting examples for use herein include classes of celluloses, acrylate polymers and acrylate crosspolymers. Preferably, hydroxypropyl cellulose, hydroxymethyl cellulose, Pluronic PF127 polymer, carbomer 980, carbomer 1342 and carbomer 940, more preferably hydroxypropyl cellulose, Pluronic PF127 carbomer 980 and carbomer 1342, more specifically hydroxypropyl cellulose (Klucel® EF, GF and/or HF), Pluronic PF127, carbomer 980 and/or carbomer 1342 (Pemulen® TR-1, TR-2 and/or Carbopol® ETD 2020).

A dermatological composition of the invention can contain one or more anti-oxidants, radical scavengers, and/or stabilizing agents, preferred concentration range from about 0.001 wt. % to about 0.1 wt. %, more preferably from about 0.1 wt. % to about 5 wt. % of the dermatological composition. Non-limiting examples for use herein include butylatedhydroxytoluene, butylatedhydroxyanisole, ascorbyl palmitate, citric acid, vitamin E, vitamin E acetate, vitamin E-TPGS, ascorbic acid, tocophersolan and propyl gallate. More specifically the anti-oxidant can be ascorbyl palmitate, vitamin E acetate, vitamin E-TPGS, vitamin E or butylatedhydroxytoluene.

A dermatological composition of the invention can also contain preservatives that exhibit anti-bacterial and/or anti-fungal properties. Preservatives can be present in a gelled dermatological composition of the invention to minimize bacterial and/or fungal over its shelf-life. Preferred concentration range of preservatives in a dermatological composition of the invention can be from about 0.001 wt. % to about 0.01 wt. %, more preferably from about 0.01 wt. % to about 0.5 wt. % of the dermatological composition. Non-limiting examples for use herein include diazolidinyl urea, methylparaben, propylparaben, tetrasodium EDTA, and ethylparaben. More specifically the preservative would be a combination of methylparaben and propylparaben.

A dermatological composition can optionally include one or more chelating agents. As used herein, the term “chelating agent” or “chelator” refers to those skin benefit agents capable of removing a metal ion from a system by forming a complex so that the metal ion cannot readily participate in or catalyze chemical reactions. The chelating agents for use herein are preferably formulated at concentrations ranging from about 0.001 wt. % to about 10 wt. %, more preferably from about 0.05 wt. % to about 5.0 wt. % of the dermatological composition. Non-limiting examples for use herein include EDTA, disodium edeate, dipotassium edeate, cyclodextrin, trisodium edetate, tetrasodium edetate, citric acid, sodium citrate, gluconic acid and potassium gluconate. Specifically, the chelating agent can be EDTA, disodium edeate, dipotassium edate, trisodium edetate or potassium gluconate.

The dermatological compositions of this invention can be provided in any cosmetically suitable form, preferably as a lotion or a cream, but also in an ointment or oil base, as well as a sprayable liquid form (e.g., a spray that includes TOFA in a base, vehicle or carrier that dries in a cosmetically acceptable way without the greasy appearance that a lotion or ointment would have when applied to the skin).

In addition, the dermatological compositions of the invention can include one or more compatible cosmetically acceptable adjuvants commonly used, such as colorants, fragrances, emollients, humectants and the like, as well as botanicals, such as aloe, chamomile and the like.

In topically administering the dermatological compositions of the invention, the skin of the human to be treated can be optionally pre-treated (such as washing the skin with soap and water or cleansing the skin with an alcohol-based cleanser) prior to administration of the dermatological composition of the invention.

In treating dermatological disorders or conditions characterized by sebaceous gland hyperactivity, TOFA can also be administered systemically, preferably orally, to the human in need thereof in pharmaceutically acceptable compositions, as described in more detail below.

A pharmaceutical composition of the invention to be orally administered can be prepared by combining TOFA with an appropriate pharmaceutically acceptable carrier, diluent or excipient by standard methods known to one skilled in the art. Pharmaceutical compositions of the invention are formulated so as to allow TOFA contained therein to be bioavailable upon administration of the composition to a human.

A pharmaceutical composition of the invention to be orally administered may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When a pharmaceutical composition of the invention is in the form of a capsule, for example, a gelatin capsule, it may contain, in addition to materials of the above type, a liquid carrier such as polyethylene glycol or oil.

A pharmaceutical composition of the invention to be orally administered may also be in the form of a liquid, for example, an elixir, syrup, solution, emulsion or suspension. The pharmaceutical composition may also optionally contain one or more of a sweetening agent, preservatives, dye/colorant and flavor enhancer.

Liquid pharmaceutical compositions of the invention may also include one or more of the following adjuvants: sterile water, saline solution (preferably physiological saline solution), Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

A liquid pharmaceutical composition of the invention contains a therapeutically effective amount of TOFA when administered to a human in need thereof. Typically, this amount is at least 0.01% of TOFA in the composition. This amount may be varied to be between about 0.1 wt. % and about 70% of the total weight of the composition. Preferred oral pharmaceutical compositions contain TOFA at a concentration range of between about 1.0 wt. % and about 50 wt. % of the oral composition.

A pharmaceutical composition of the invention may include various materials, which modify the physical form of a solid or liquid dosage unit. For example, the composition may include materials that form a coating shell around the active ingredient. The materials that form the coating shell are typically inert, and may be selected from, for example, sugar, shellac, and other enteric coating agents. Alternatively, the active ingredient may be encased in a gelatin capsule.

A pharmaceutical composition of the invention in solid or liquid form may also include an agent that binds to TOFA and thereby assists in the systemic delivery of TOFA. Suitable agents that may act in this capacity include a monoclonal or polyclonal antibody, a protein or a liposome.

Systemic administration of the pharmaceutical compositions of the invention also include administration by injection, e.g., subcutaneous, intravenous, intramuscular, intrathecal or intraperitoneal injection, as well as transdermal, transmucosal, or pulmonary administration and needle-less injection administration.

Useful injectable pharmaceutical compositions include sterile suspensions, solutions or emulsions of the active compound(s) in aqueous or oily vehicles. The compositions may also contain formulating agents, such as suspending, stabilizing and/or dispersing agent. The pharmaceutical compositions for injection may be presented in unit dosage form, e.g., in ampules or in multidose containers, and may contain added preservatives.

Alternatively, the injectable pharmaceutical compositions may be provided in powder form for reconstitution with a suitable vehicle, including but not limited to sterile pyrogen free water, buffer, dextrose solution, etc., before use. To this end, the active compound, i.e., TOFA, may be dried by any art-known technique, such as lyophilization, and reconstituted prior to use.

For transmucosal administration, penetrants appropriate to the barrier to be permeated are used in the formulation. Such penetrants are known in the art.

For prolonged delivery, TOFA, or a pharmaceutically acceptable salt thereof, can be formulated as a depot preparation for administration by implantation or intramuscular injection. TOFA, or a pharmaceutically acceptable salt thereof, may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases TOFA, or a pharmaceutically acceptable salt thereof, for percutaneous absorption may be used. To this end, permeation enhancers may be used to facilitate transdermal penetration of the active compound(s). Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Administration of the pharmaceutical compositions of the invention by needle-less injection can be employed using the techniques disclosed in U.S. Pat. No. 6,756,053.

Alternatively, other pharmaceutical delivery systems may be employed for the pharmaceutical compositions of the invention. Liposomes and emulsions are well-known examples of delivery vehicles that may be used to deliver active compound(s) or prodrug(s). Certain organic solvents such as dimethylsulfoxide (DMSO) may also be employed, although usually at the cost of greater toxicity.

The pharmaceutical compositions of the invention may, if desired, be presented in a pack or dispenser device which may contain one or more unit dosage forms containing the active compound(s). The pack may, for example, comprise metal or plastic foil, such as a blister pack. The pack or dispenser device may be accompanied by instructions for administration.

The pharmaceutical compositions of the invention as set forth above may be prepared by methodology well known in the pharmaceutical art or by the method described herein. See, for example, Remington's Pharmaceutical Sciences, 18th Ed., (Mack Publishing Company, Easton, Pa., 1990).

The pharmaceutical compositions of the invention are administered to a human in a therapeutically effective amount, which will vary depending upon a variety of factors including the activity of TOFA; the metabolic stability and length of action of the TOFA; the age, body weight, general health, sex, and diet of the human; the mode and time of administration; the rate of excretion; the drug combination; and the severity of the particular disorder or condition. Generally, a therapeutically effective daily dose of TOFA is (for a 70 kg mammal) from about 0.001 mg/kg (i.e., 0.07 mg) to about 100 mg/kg (i.e., 7.0 gm); preferably a therapeutically effective dose is (for a 70 kg mammal) from about 0.01 mg/kg (i.e., 0.7 mg) to about 50 mg/kg (i.e., 3.5 gm); more preferably a therapeutically effective dose is (for a 70 kg mammal) from about 1 mg/kg (i.e., 70 mg) to about 25 mg/kg (i.e., 1.75 gm).

The following Examples 5-9 provide dermatological compositions of the invention comprising TOFA and one or more dermatologically acceptable excipients.

### EXAMPLE 5

### TOFA Dermatological Alcoholic Gel Formulation

The product of the following formulation is a semi-solid clear gel.

| Ingredient | Percent w/w |
|---|---|
| TOFA | 1.0 |
| Diethylene Glycol Monoethyl Ether, NF | 32.0 |
| Tocophersolan, NF | 1.0 |
| Hydroxypropyl Cellulose, NF (Klucel ® GF) | 4.0 |
| Edetate Disodium | 0.05 |
| Alcohol, Dehydrated, NF | 61.95 |

Method of Manufacture: The alcohol and diethylene glycol monoethyl ether are combined. Tocophersolan, edetate disodium and TOFA are dissolved with mixing. Hydroxypropyl cellulose is added and quickly and evenly dispersed with high-speed mixing. The product is removed from mixing after uniform dispersion.

### EXAMPLE 6

### TOFA Dermatological Aqueous Gel Formulation

The product of the following formulation is a semi-solid clear soft gel.

| Ingredient | Percent w/w |
|---|---|
| TOFA | 1.0 |
| Diethylene Glycol Monoethyl Ether, NF | 30.0 |
| Glycerin, USP | 5.0 |
| Tocophersolan, NF | 1.0 |
| Methylparaben, NF | 0.1 |
| Propylparaben, NF | 0.02 |
| Edetate Disodium | 0.05 |
| Acrylates/C10-C30 Alkyl Acrylate | 2.0 |
| Crosspolymer, NF | |
| Polysorbate 80, NF | 0.1 |
| Trolamine, NF | to pH 6.75 |
| Water, USP | to 100.0 |

Method of Manufacture: The liquids, diethylene glycol monoethyl ether, glycerin and water, are mixed. Polysorbate 80 and tocophersolan are added and mixed to dissolve. TOFA is added and mixed to dissolve. Edetate disodium, methylparaben, and propylparaben are added and mixed to dissolve. Acrylates/C10-C30 alkyl acrylate crosspolymer are quickly dispersed with high-speed mixing until uniform mixture obtained. Trolamine is added with constant mixing to obtain a viscous gel at a pH of approximately 6.75 (when diluted 1:9 with water).

### EXAMPLE 7

### TOFA Dermatological Hydroalcoholic Gel Formulation

The product of the following formulation is a semi-solid clear soft gel.

| Ingredient | Percent w/w |
|---|---|
| TOFA | 1.0 |
| Diethylene Glycol Monoethyl Ether, NF | 30.0 |
| Alcohol, NF | 25.0 |
| Glycerin, USP | 5.0 |
| Tocophersolan, NF | 1.0 |
| Methylparaben, NF | 0.1 |
| Propylparaben, NF | 0.02 |
| Edetate Disodium | 0.05 |
| Hydroxypropyl Cellulose, NF (Klucel ® EF) | 2.0 |
| Acrylates/C10-C30 Alkyl Acrylate | 1.0 |
| Crosspolymer, NF | |
| Polysorbate 80, NF | 0.05 |
| Trolamine, NF | to pH 6.75 |
| Water, USP | to 100.0 |

Method of Manufacture: The liquids, diethylene glycol monoethyl ether, glycerin alcohol and water, are mixed. Polysorbate 80 and tocophersolan are added and mixed to dissolve. TOFA is added and mixed to dissolve. Edetate disodium, methylparaben and propylparaben are added and mixed to dissolve. Acrylates/C10-C30 alkyl acrylate crosspolymer and hydroxypropyl cellulose are quickly dispersed with high-speed mixing until uniform mixture obtained. Trolamine is added with constant mixing to obtain a viscous gel at a pH of approximately 6.75 (when diluted 1:9 with water).

### EXAMPLE 8

### TOFA Dermatological Cream Formulation

TOFA may also be formulated as a cream, an example of which is as follows:

| Ingredient | Percent w/w |
|---|---|
| TOFA | 1.0 |
| Diethylene Glycol Monoethyl Ether, NF | 20.0 |
| White Petrolatum | 5.0 |
| Isopropyl Myristate | 5.0 |
| Cetostearyl Alcohol | 5.0 |
| Trilaureth-4 Phosphate | 1.0 |
| Tocophersolan, NF | 1.0 |
| Cyclomethicone, NF | 5.0 |
| Methylparaben, NF | 0.2 |
| Propylparaben, NF | 0.04 |
| Edetate Disodium | 0.05 |
| Carbomer 940 | 0.15 |
| Acrylates/C10-C30 Alkyl Acrylate | 0.15 |
| Crosspolymer, NF | |
| Trolamine, NF | to pH 6.75 |
| Water, USP | to 100.0 |

Method of Manufacture:

A. Water Phase

Water and diethylene glycol monoethyl ether are mixed together. Tocophersolan is added and mixed to dissolve. TOFA is added and mixed to dissolve. Trilaureth-4 phosphate, edetate disodium, methylparaben and propylparaben are added and mixed to dissolve. Acrylates/C10-C30 alkyl acrylate crosspolymer and carbomer 940 are quickly dispersed with high-speed mixing until uniform mixture obtained. The resulting mixture is heated, while stirring, at a temperature of between about 65° C. and about 75° C. to form a solution.

B. Oil Phase

White petrolatum, cyclomethicone, isopropyl myristate and cetostearyl alcohol are combined in a separate vessel and melted completely at a temperature of between about 65° C. and about 75° C. and stirred.

C. While stirring the water phase, the oil phase is slowly added until a uniform emulsion is obtained. Trolamine is slowly added to the resulting emulsion to obtain a cream at a pH of approximately 6.75. The product is cooled to 25° C. with continuous mixing.

### EXAMPLE 9

### TOFA Dermatological Foam Formulation

TOFA may also be formulated as a foam, an example of which is as follows:

| Ingredient | Percent w/w* |
|---|---|
| TOFA | 1.0 |
| Diethylene Glycol Monoethyl Ether, NF | 25.0 |
| Stearyl Alcohol, NF | 8.0 |
| Laureth-23 | 0.5 |
| PEG-100 Stearate | 1.0 |
| Tocophersolan, NF | 1.0 |
| Propylparaben, NF | 0.3 |
| Edetate Disodium | 0.05 |
| Acrylates/C10-C30 Alkyl Acrylate | 0.2 |
| Crosspolymer, NF | |
| Trolamine, NF | to pH 6.75 |
| Water, USP | to 100.0 |

*Propellant is 4.0 wt. % of final formulation. The propellant is a single gas or a mixture of gases. Suitable gases include butane, isobutane, propane, isopropane and isopentate.

Method of Manufacture:

A. Water Phase

Water and diethylene glycol monoethyl ether are mixed. Tocophersolan is added and mixed to dissolve. TOFA is added and mixed to dissolve. Edetate disodium and propylparaben are added and mixed to dissolve. Acrylates/C10-C30 alkyl acrylate crosspolymer is quickly dispersed with high-speed mixing until uniform mixture obtained. The resulting mixture is heated, while stirring, to solution at a temperature of between about 60° C. and about 70° C.

B. Oil Phase

Stearyl alcohol, laureth-23 and PEG-100 stearate are combined in a separate vessel and melted completely. while stirring, at a temperature of between about 60° C. and about 70° C.

C. While stirring the water phase, the oil phase is added until a uniform emulsion is obtained. Trolamine is added to afford the desired pH. The resulting formulation is cooled to 25° C. with continuous mixing. The formulation is packaged in an appropriate air-tight container under pressure with propellant.

Combination Therapy

TOFA may be usefully combined with one or more other therapeutic agents in the treatment of dermatological disorders or conditions characterized by sebaceous gland hyperactivity. For example, TOFA may be administered simultaneously, sequentially or separately in combination with other therapeutic agents, including, but not limited to:
topical/oral antibiotics, e.g., clindamycin, tetracycline, minoccline, deoxycycline, erythromycin, trimethoprim, and azithromycin;
retinoids, e.g., Accutane®, tretinion, tazarotene, and adapalene;
benzoyl peroixide;
blue/red light;
photodynamic therapy (PDT);
Anti-androgenic compounds, e.g., PSK 3841;
5-alpha reductase type I inhibitors;
comedolytics, e.g., salicylic acid, azelaic acid, sulfur and resorcinol.

As used herein “combination” refers to any mixture or permutation of TOFA and one or more additional therapeutic agents useful in the treatment of dermatological disorders or conditions. Unless the context makes clear otherwise, “combination” may include simultaneous or sequentially delivery of TOFA with one or more therapeutic agents. Unless the context makes clear otherwise, “combination” may include dosage forms of TOFA (e.g., dermatological or pharmaceutical compositions comprising TOFA and a dermatological acceptable excipient) with another therapeutic agent. Unless the context makes clear otherwise, “combination” may include routes of administration of TOFA with another therapeutic agent. Unless the context makes clear otherwise, “combination” may include compositions comprising TOFA and another therapeutic agent. Dosage forms, routes of administration and dermatological and pharmaceutical compositions include, but are not limited to, those described herein.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

What is claimed is:

1. A method of treating a human having a disorder or condition characterized by inflammation, wherein the method comprises administering to the human in need thereof a pharmaceutical composition comprising a therapeutically effective amount of 5-(tetradecyloxy)-2-furancarboxylic acid, or a pharmaceutically acceptable salt thereof, and a pharmaceutically acceptable excipient.

2. The method of claim 1, wherein the disorder or condition is inflammatory acne.

3. The method of claim 1, wherein the therapeutically effective amount of 5-(tetradecyloxy)-2-furancarboxylic acid, or a pharmaceutically acceptable salt thereof, is administered topically.

4. The method of claim 1, wherein the therapeutically effective amount of 5-(tetradecyloxy)-2-furancarboxylic acid, or a pharmaceutically acceptable salt thereof, is administered systemically.

5. The method of claim 4, wherein the therapeutically effective amount of 5-(tetradecyloxy)-2-furancarboxylic acid, or a pharmaceutically acceptable salt thereof, is administered orally.

6. The method of claim 1, wherein the pharmaceutical composition is a dermatological composition and the pharmaceutically acceptable excipient is a dermatologically acceptable excipient.

7. The method of claim 1, wherein the therapeutically effective amount of 5-(tetradecyloxy)-2-furancarboxylic acid reduces T cell proliferation and cytokine secretion in the human.
